(19) United States
(12) Patent Application Publication
(10) Pub. No.: US 2017/0231891 A1
(43) Pub. Date: Aug. 17, 2017

(54) SULFATED POLYSACCHARIDES WITH INHERENT ANTIMICROBIAL PROPERTIES, AND A METHOD OF MANUFACTURING THE SAME

(71) Applicant: Robert Falken, Oceanside, CA (US)

(72) Inventor: Robert Falken, Oceanside, CA (US)

(21) Appl. No.: 15/138,182

(22) Filed: Apr. 25, 2016

Related U.S. Application Data
(60) Provisional application No. 62/294,263, filed on Feb. 11, 2016.

(51) Int. Cl. A61K 8/73; A61Q 15/00; A01N 25/10; A61K 8/85; A61K 8/37; A01N 65/03; A61K 8/06; A61Q 1/06

(52) U.S. Cl. CPC A61K 8/73; A61K 8/064; A61Q 15/00; A61K 8/06; A61Q 1/06; A61K 8/85; A61K 8/37; A01N 65/03; A01N 25/10; A61K 2800/74

(57) ABSTRACT

This document presents sulfated polysaccharides derived from renewable non-terrestrial plants exhibiting exceptional antimicrobial properties and characteristics, and a method of manufacturing the same. Any of various species of red algae, brown algae, and brown seaweed (marine microalgae and/or macroalgae) are known to contain a high level of sulfated polysaccharides with inherent antimicrobial properties, and can be used in various implementations.

### CROSS-REFERENCE TO RELATED APPLICATION

This application claims the benefit of U.S. Provisional Application Ser. No. 62/294,263, filed on Feb. 11, 2016 and titled “SULFATED POLYSACCHARIDES WITH INHERENT ANTIMICROBIAL PROPERTIES, AND A METHOD OF MANUFACTURING THE SAME,” the disclosure of which is incorporated by reference in its entirety.

### BACKGROUND

This document presents sulfated polysaccharides derived from renewable non-terrestrial plants exhibiting exceptional antimicrobial properties and characteristics.

It is a longstanding requirement of industry that products exhibit antimicrobial and antifungal performance. Using metallic particles as antimicrobial and antifungal agents is known, but success has been challenging due to high material cost, oxidation during use, limited global supply, as well as significant concerns regarding the potential negative health and environmental consequences from their use. For these reasons, there exists a continuing and unmet need for improved antimicrobials derived from renewable non-metallic natural sources.

### SUMMARY

This document presents sulfated polysaccharides derived from renewable non-terrestrial plants exhibiting exceptional antimicrobial properties and characteristics, and a method of manufacturing the same. Any of various species of red algae, brown algae, and brown seaweed (marine microalgae and/or macroalgae) are known to contain a high level of sulfated polysaccharides with inherent antimicrobial properties, and can be used in various implementations of the subject matter described herein.

The details of one or more embodiments are set forth in the accompanying drawings and the description below. Other features and advantages will be apparent from the description and drawings, and from the claims.

### BRIEF DESCRIPTION OF THE DRAWINGS

These and other aspects will now be described in detail with reference to the following drawings.

FIG. 1 is a flowchart of a non-limiting method for extracting one or more sulfated polysaccharides from the cell walls of renewable non-terrestrial plants having exceptional antimicrobial and/or antifungal properties;

FIG. 2 is a graph comparing the CFU/Carrier vs Concentration over Time for Test 1.

FIG. 3 is a graph comparing the CFU/Carrier vs Concentration over Time for Test 2.

Like reference symbols in the various drawings indicate like elements.

### DETAILED DESCRIPTION

This document presents sulfated polysaccharides derived from renewable non-terrestrial plants exhibiting exceptional antimicrobial properties and characteristics, and a method of manufacturing the same. Said plants can best be described as one or more types of algae (marine microalgae and/or macroalgae). As a feedstock, algae are a suitable solution for making antimicrobials due in part to its significant abundance worldwide. In addition to significantly inhibiting bacteria growth without harm to human health, algae offer many environmental benefits for use in manufacturing antimicrobials, such as requiring no pesticides to grow, sequestering C02, and not adversely affecting the environment.

Algae are plantlike protists, which are eukaryotic, unicellular or multicellular organisms. Like plants, algae have chloroplasts, and their cells are strengthened by a cell wall. Algae refer to aquatic organisms that carry on photosynthesis, and are typically part of a fresh water system's phytoplankton. Algae may be classified by pigmentation, such as green Chlorophyta, brown Phaeaphyta, golden brown Chrysphyta, and red Rhodophyta for example.

Although all algae share various typical physical traits, so as to appear to be related, in many instances various members of the group commonly referred to as algae are in fact unrelated and/or are derived from multiple ancestral sources. In particular, algae may be unicellular or multicellular, but in most instances are aquatic. Algae derive their energy from photo or chemo-synthesis and tend to be self-nourishing organisms that are capable of producing complex compounds such as carbohydrates, fats, and proteins, such as from simple substances present in their local environments. Specifically, algae are capable of absorbing carbon dioxide in their production of organic compounds that themselves may be employed in biosynthesis, e.g., using water or hydrogen sulfide as a reducing agent, and/or for the storage of chemical energy. Hence, various algae can convert electromagnetic energy, such as from sunlight, into a chemical energy, such as in the form of reduced carbon.

Particularly, multicellular forms of algae include kelps, such as giant kelp, and brown alga. For instance, brown algae are a large group of multicellular, marine organisms that are typically referred to as seaweeds. Seaweeds are plantlike organisms that typically live attached to the surfaces of rocks or other hard substrata along coastal areas. More particularly, seaweed includes red, brown, and green algae, as described above. However, even though these marine based organisms bare the common name of seaweed they are not derived from a common ancestor, and thus, do not share a common heritage.

Nevertheless, the various members of seaweed do tend to share several phenotypical traits or structures. For example, they typically have a main body or thallus that forms a stem-like structure called a stipe. They also include a plurality of blade structures that are somewhat flattened, leaf-like structures. It is on the basis of the color of the thallus by which seaweeds are distinguished as brown, red, and green algae. Brown and red algae are commonly found in saltwater environments, while green algae are typically found in freshwater. The predominance of the brown color in brown algae is derived from a xanthophyll pigment, e.g., fucoxanthin, and beta-carotene. These algae generally produce complex polysaccharides, sugars, and alcohols. Examples of brown algae are Ascophyllum, Fucus, Laminaria, Macrocystis, Saccharina, and Undaria. Specific examples include kelp, limu, laminaria, vesiculosus, and wakame. Brown Algae may further be catalogued as brown seaweed, and may include such diverse members as mozuku, kombu, bladderwrack, and hijiki. Fucus vesiculosus, for instance, is a brown seaweed that is composed of a simple chemical composition of fucose and sulfate, but may also include other simple sugars such as mannose, galactose, glucose, xylose, and the like, as well as uronic acids and proteins. Other specific brown seaweeds include Chorda filum, Ecklonia kurome, F. evanescens, F. distichus, F. serratus, F. vesiculosus, and Macrocytis pyrifera.

The predominance of the red color in red algae is derived from a phycoerthrin and phycocyanin pigments, as well as beta-carotene, and other xanthophils. These algae generally produce cellulose, long-chained polysaccharide, agars, and carrageenans. Examples of red algae are Rhodophyta, Kappaphycus, Betaphycus, Corallinaceae, Gigartinaceae, Gracilaria, Gelidium, and Pterocladia. The predominance of the green color in green algae is derived from chlorophyll a and b pigments, as well as beta-carotene and other xanthophils These algae generally produce fats and oils. Examples of green algae are Bryopsidophytes, Caulerpa, Charophytes, Chlorophytes, e.g., Chlorella, Dunaliella, Dasycladophytes, Desmids, Siphoncladophytes, Spirogyra, Trebouxiophytes, and Ulvophyceae.

The blade further includes a plurality of air bladders, which function to assist in allowing the seaweed to float. A stipe, or stem-like structure may also be present, and typically the air balder is present between the blade and the stipe. Together the blade and stipe from a fond. In aquatic environments, a holdfast, e.g., a basal structure, may be present, so as to provide attachment to local surfaces, e.g., to prevent the seaweed from being washed out to sea. The holdfast will typically include fingerlike extensions so as to better anchor the seaweed to various rocks and surfaces within the aquatic environment.

Today, seaweeds themselves are used in many products, including those that serve as fodder, fertilizers, and food, and dependent on its mode of manufacture may be used as alternative fuel sources and/or biochemical. Particularly, seaweeds typically grow in seawater (or freshwater, e.g., for green algae), such as by deriving energy from the sun and absorption of carbon dioxide, and may then be processed to give off oxygen, be subjected to nutrient scrubbing, such as to give off a useful effluent. Such algae as herein described may be subjected to extraction, such as for the production of various biochemicals; fermentation, such as for use in the production of alcohols and methane gas; and/or pyrolysis, such as for the production of fuels, such as gas.

More particularly, various of these algae, or component parts thereof may be used as human foods, or food products, such as agars, carrageenans, alginates, gums, and the like, as well as in bath products, tooth pastes, toothbrushes, cosmetics, and/or in medicinal uses. For instance, the main components of various algae include water, carbohydrates, such as fucose, proteins, and fats, also sulfates, ash, alginic acid, xylans, laminaran, mannitol, Fucoidan, floriside, tannins, sodium, magnesium, iodine, and other components. For example, see Table I.

TABLE I

| Brown seaweed | Chemical composition |
|---|---|
| Adenocytis utricularis | fucose, galactose, mannose, sulfate |
| Ascophyllum nodosum | fucose, xylose, GlcA, sulfate |
| Bifurcaria bifurcate | fucose, xylose, GlcA, sulfate |
| Dictyota menstruali | fucose/xylose/uronic |
| | acid/galactose/sulfate |
| Ecklonia kurome | fucose, galactose, mannose, xylose, |
| | GlcA, sulfate |
| F. distichus | fucose/sulfate/acetate |
| F. evanescens | fucose/sulfate/acetate |
| F. serratus | fucose/sulfate/acetate |
| F. vesiculosus | fucose, sulfate |
| Himanthalia lorea | fucose, xylose, GlcA, sulfate |
| Hizikia fusiforme | fucose, galactose, mannose, xylose, |
| | GlcA, sulfate |
| Laminaria angustata | fucose/galactose/sulfate |
| Lessonia vadosa | fucose, sulfate |
| Macrocytis pyrifera | fucose/galactose/sulfate |
| Padina pavonia | fucose, xylose, mannose, glucose, |
| | galactose, sulfate |
| Pelvetia wrightii | fucose/galactose/sulfate |
| Sargassum stenophyllum | fucose, galactose, mannose, GlcA, |
| | glucose, xylose, sulfate |
| Spatoglossum schroederi | fucose/xylose/galactose/sulfate |
| Undaria pinnatifida | fucose/galactose/sulfate |

Hence, as can be seen with respect to Table I, fucose is a component constituent of many forms of brown seaweed. Specifically, there are at least two distinct forms of fucoidan: F-fucoidan and U-fucoidan. F-fucoidan is basically composed of sulfated esters of fucose (e.g., about >95%), and U-fucoidan is about 20% glucuronic acid. These different forms are important when configuring brown algae and/or its constituents for industrial and/or commercial use. In particular instances, brown algae and its components, e.g., fucoidan, may include various sulfanates, such as sulfated polysaccharides, sulfated galactofucants, sulfated galactans, and the like.

Accordingly, any of various species of red algae, brown algae, and brown seaweed (marine macroalgae or microalgae) are known to contain high levels of sulfate, such as sulfated polysaccharides, which sulfates have been determined by the inventor hereof to have antimicrobial properties, and can be used in various implementations of the subject matter described herein. An example of a beneficial algal-derived sulfated polysaccharides for use in some implementations is dried fucoidan powder. Fucoidan designates a group of certain fucose-containing sulfated polysaccharides (FCSPs) and is not found in terrestrial plants. It has a backbone built of (1→3)-linked α-1-fucopyranosyl or of alternating (1→3)- and (1→4)- linked α-1-fucopyranosyl residues, but also include sulfated galactofucans with backbones built of (1→6)-β-d-galacto- and/or (1→2)-β-d-mannopyranosyl units with fucose or fucooligosaccharide branching, and/or glucuronic acid, xylose or glucose substitutions. There are at least two distinct forms of fucoidan: F-fucoidan, which is >95% composed of sulfated esters of fucose, and U-fucoidan, which is approximately 20% glucuronic acid. Fucoidan largely contains sulphated L-fucose residues. Hence, fucose is the primary sugar found in fucoidan. Sulphate groups also represent a large component of fucoidan and the biological activities of fucoidan are strongly related to sulphate. Besides fucose and sulphate, other monosaccharides (glucose, mannose, galactose, xylose, etc), uronic acids, and even protein are present in detectable amounts, though the total composition will vary between species, extraction methods, and environmental conditions.

For instance, Fucoidan derived from brown algae may typically be composed of about 44.1% fucose, 26.3% sulfate and 31.1% ash and/or may contain acetate and other constituents. It is to be noted that the structures and chemical constituents of various fucoidans from different brown algae vary from species to species. Specifically, as indicated Fucoidan is a fucose-containing sulfated polysaccharides that has a backbone of linked fucopyranosyl residues and may include an additional backbone of sulfated galactofucans and mannopyranosyl. The difference in backbone structures of various fucoidans reflects the fundamental difference in fucoidans biosynthesis within the various organisms form which they are produced. Particularly, fucose is a hexose deoxy sugar having a chemical formula of C₆H₁₂O₅. It includes the following chemical structure:

Fucose is the fundamental sub-unit of the fucoidan polysaccharide. It is to be noted that fucose includes an L-configuration that lacks a hydroxyl group on the carbon at the 6-position (C-6), making it a deoxy sugar. Accordingly, Fucoidan is a polysaccharide that includes a substantial percentage of L-fucose and sulfate ester groups. For instance, ordered fucoidans may contain a linear backbone built up of (1→3)-α-L-Fuc or alternating (1→3)-α-L-Fuc and (1→4)-α-L-Fuc, (1→2)-α-L-Fuc sometimes being present in the backbone branching. Sulfate groups often occupy the C-2 or/and C-3, C-4 of fucose. For fucoidans containing uronic acid (UA) and hexose, the structural core may be built of alternating UA-hexose. This structure is very stable because of this and other sugars present in the branching off at the core backbone.

Hence, fucoidan may also include fucose and/or fucooligosaccharide branching, and may include glucuronic acid, xylose, and various glucose substitutions. Specifically, the core region of Fucoidan is primarily a polymer of α-(1→3) linked fucose with sulfate groups substituted at the C-4 position on some of the fucose residues; fucose may also be attached to this polymer to form branched points, e.g., one for every 2-3 fucose residues within the chain. More specifically, some types of Fucoidan may be composed of a linear backbone of alternating 3- and 4-linked α-L-fucopyranose 2-sulfate residues: →3)-α-L-Fucp(2SO3-)-(1→4)-α-L-Fucp(2SO3-)-(1→, with additional sulfates occupying position 4 in a part of 3-linked fucose residues, whereas a part of the remaining hydroxyl groups may randomly be acetylated.

Other types of Fucoidan may be built up of disaccharide repeating units: →3)-a-L-Fucp-(2,4-di-SO3-)-(1→4)-α-L-Fucp-(2SO3-)-(1→, such as where the regular structure may be only slightly masked by random acetylation and undersulfation of several disaccharide repeating units. In other instances, fucoidan may have a branched structure, such as where its backbone is →3)-α-L-Fucp-(1→4)-α-L-Fucp-(1→, about half of the 3-linked residues may be substituted at C-4 by α-L-Fucp-(1→4)-α-L-Fucp-(1→3)-α-L-Fucp-(1→trifucoside units. Sulfate groups may also occupy mainly C-2 and sometimes C-4, although 3,4-diglycosylated and some terminal fucose residues may be nonsulfated. Acetate groups may occupy C-4 of 3-linked Fuc and C-3 of 4-linked Fuc, such as in a ratio of about 7:3. Fucoidan may also contain small amounts of xylose and galactose. In further instances, sulfated fucan may have a backbone of (1→4)- and (1→3)-linked-α-L-fucopyranosyl residues that are substituted at C-2 and C-3, and fucosyl residues may be sulfated at C-2 and/or C-4. Additionally, Fucoidan may be composed of one or more well formed polycrystalline ultrastructures that include sulfated fucan. Such particles may further be constituted by sulfated fucan molecules that are composed partially of a lectin specific for α-L-fucosyl residues.

Hence, as can be seen, the chemical compositions and structures of fucoidans from brown algae are very complex and their structures vary from species to species. Its bioactive properties vary depending on the source from which it is derived, its compositional and structural traits, and its purity. For instance, in various instances, the position of the sulfate groups may be important to various biological activities of fucoidan containing sulfated polysaccharides. For example, most sulfate groups in fucoidan are in axial positions, while the remainder are in equatorial positions. Particularly, the preservation of the structural integrity of the fucoidan molecules essentially depends on its method of extraction, which has an important significance for obtaining the relevant structural features required for specific biological activities based on its various structure-function relations.

Any suitable sulfated polysaccharide derived from freshwater algae or saltwater algae (seaweed) may be utilized in accordance with the present disclosure. Additionally, marine organisms such as sea cucumbers and sea urchins have been found to contain some levels of sulfated polysaccharides, and as such, could potentially be utilized as a feedstock of the present invention. However, the fucoidan and the polysaccharides from the aforementioned marine organisms are not simple compounds; they are complex mixtures of many carbohydrate structures, and as such further research is required to determine their antimicrobial effectiveness. Hence, due to the complexity of its chemical composition and structure, the difficulty in its extraction and processing, as well as the fact that its structures and chemical make up vary widely from species to species, industrial use and commercial viability have been largely limited.

Particularly, the use of fucoidan is largely limited for the production of oil based products, where isolation and extraction of its component parts is not of primary concern. For instance, the inclusion of complex carbohydrates, proteins, and lipids in the blade and/or air bladders of the algae make the extraction and processing of fucoidan difficult. Nevertheless, it has been determined by the inventor hereof that brown algae and various of its constituent components, when isolated and processed appropriately may be useful for many commercially viable products. Specifically, due to the research performed by the inventor herein, Fucoidan, and/or its compositional components may have many uses, such as for food packaging, such as for the production of films, substrates for contact with meats, disinfectant sprays, plastics, and the like.

For instance, fucoidan and its constituents, such as various sulfates, may be derived from several different sources, such as from fresh water algae, e.g., green algae, or salt water algae, e.g., brown and red algae, as well as from other macro/micro algae, sea cucumbers, and sea urchins. For example, fucoidan can be extracted from crude raw materials, and be purified by any suitable mechanism, such as ion-exchange chromatography, gel filtration, and/or by using a solvent, such as by water or other solvents, such as hydrochloric acid, calcium chloride, and/or acid/base extraction (particularly used for raw seaweed extraction). In particular instances, water may be used as a solvent, such as to maintain bioactivity, ensure a stable molecular weight, and maintain the charge of the various constituent polysaccharides.

Particulalry, in an exemplary method for isolating and/or purifying fucoidan and/or various of its constituent components from algae, a first step may include growing and/or harvesting or otherwise collecting a quantity of the algae from its source, such as from a salt or freshwater pool or other farm. Other steps may include one or more of crushing and/or further processing the algae so as to separate its cellular constituents from the structural matrix that forms the organism. The algal cells may then be dispersed and/or separated such as by agitation and/or by steaming in a high pressure, high temperature water container, such as from 80 to 90 degrees Celsius, such as for about 60 to about 90 minutes. Alcohol may optionally be added to facilitate the extraction and/or separation process. If alcohol is used, it may be removed after processing therewith, such as by an extraction tank. The resultant composition may then be filtered, concentrated, and purified.

Once isolated, the fucoidan may be dried and/or milled to a desired particle size. For instance, in various instances, various constituents of ficoidan may be isolated and separated therefrom by one or more of these or associated processes, such as to isolate various sulfates, such as sulfated polysaccharides, sulfated galactofucans, sulfated galactans, and the like. In particular instances, once isolated, dried, and formed into a powder, the powder may be further processed so as to separate the powder into pre-selected size ranges, such as where the average particle size may have a diameter range from about 2 to about 12 micrometers, such as from about 4 to about 10 micrometers, for instance, from about 6 to about 8 micrometers, including a particle having a mean diameter of about 5 micrometers. Particularly, in some instances, the particulate composition may have a moisture content of about 0 to about 15%, such as from about 2 to about 12%, for instance, about 4 to about 10%, including about 5%.

Once in powder form, the ficoidan derived extracts may be blended with one or more agents, as described herein below, so as to be formulated into various constituents for the production of several useful products, including plastics and foams, personal health care and/or cosmetic products, and the like, which may then be used for the manufacturing of particular industrial and/or commercial products such as personal care products, such as deodorants, toothpastes, toothbrushes and bristles, floss, combs, hair brushes, and the like. Additionally, it may be formulated into various cosmetics such as mascara, eyeliner, blushes, lipsticks, and the like. Additionally, it may be formed as a film or substrate and used for food packaging, such as to contact meat, it may be aerosolized and used as a disinfectant spray, and/or may be foamed or otherwise may into a plastic.

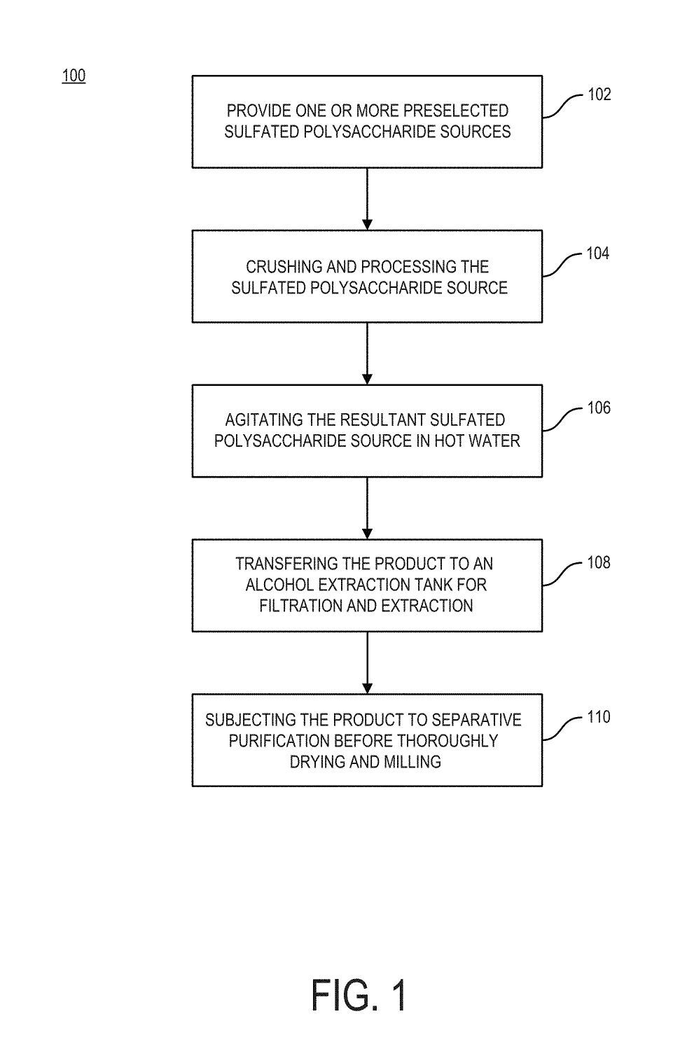

Accordingly, in one apsect, as generally illustrated in FIG. 1, methods are provided for extracting sulfated polysaccharides from the cell walls of renewable non-terrestrial plants having exceptional antimicrobial and/or antifungal properties. Various extraction methods may be employed to produce and preserve high quality fucoidan. The primary extraction methods utilized by industry tend to extract fucoidan as a multicomponent crude form of 15 fucoidan, commonly called crude fucoidan. In order to obtain purified fucoidan, ion-exchange chromatography or gel filtration can be applied to crude fucoidan. Raw seaweeds are usually extracted with acid/base solutions as the solvent, though water is now frequently used to extract crude fucoidan as it maintains the stability of the molecular weight and overall charge of the polysaccharide. Using water as the solvent is critical in producing high quality fucoidan. Additionally, the use of water for extraction ensures that the extracted material retains its natural bioactivity.

Three extracting solvents normally used to extract fucoidan are deionized water, hydrochloric acid, and calcium chloride (CaCl2). A useful non-limiting method of extraction in this invention is water extraction. For instance, a water extraction method may be employed to extract fucoidan. This non-limiting method of fucoidan extraction begins with the collection of fresh brown algae, crushing and processing said brown algae, separating the algae cells in an agitating hot water extractor or high pressure extraction pot for 60-120 minutes at 80-90° C., then adding alcohol to the viscous extract (as occasion demands) where the boiled product is transferred to an alcohol extraction tank, cleared of the alcohol and subjected to filtration and extraction to give a stock solution. Said stock solution is then subjected to separative purification and concentration before being thoroughly dried. The outcome is dried fucoidan powder that can be milled to a desired particle size that exhibits exceptional antimicrobial properties for inclusion into consumer and/or industrial products.

Hence, in one aspect of the present disclosure, is provided useful compositions including fucoidan and/or its extracts that may be included in various different products, such as to add many beneficial qualities that the underlying product would not itself contain or otherwise exhibit. Particularly, fucoidan and various of its extracts include very beneficial properties, which in some instances, include anti-microbial properties, which properties may be exhibited by the various products to which the fucoidan and/or its extracts are added, such as during the fabrication process. For instance, such fucoidan and/or its extracts may be added to various different compositions such as in the formulation of various foams, resins, plastics, including thermoplastics (e.g., without plasticizers), fibers, and the like.

More particularly, fucoidan and/or its extracts may be added to various compositions such as to impart anti microbial properties thereto, such as to inhibit bacterial growth. For example, fucoidan and various of its extracts have been shown to reduce about 80 to about 99% of the growth of gram positive and/or gram negative bacterial growth, such as Staphylococcus aureus and Escherichia coli. In such instances, the fucoidan may be part of the underlying composition or may be coated onto a surface of the product such as via a suitable treatment process. In particular instances, fucoidan and/or its extracts, such as sulfated polysaccharides, sulfated galactofucans, and/or sulfated galactans may be added to a composition, such as during a fabrication process, or to a surface of a finished product, such as after fabrication so as to add anti-microbial properties thereto.

Hence, in various embodiments, an antimicrobial powder, such as derived from algae, containing a fucoidan extract, such as a sulfate, such as a sulfated polysaccharide, sulfated galactofucans, sulfated galactans, and/or the like, may be provided, such as to a composition, such as during a fabrication process, so as to depart antimicrobial properties to the underlying article of manufacture. Particularly, such antimicrobial agents may be included in the fabrication of various plastics, so as to depart antimicrobial properties thereto. In such instances, plastics fabricated in the manners provided herein have several advantages over those not containing or otherwise contacted with the ficoidan and/or its extracts, which include non-clumping, non-agglomeration, and wont separate out of solution during the fabrication process.

For instance, during the fabrication process a liquid comprising one or more of the reactants disclosed herein and/or a plastic precursor and/or a block copolymer may be added, e.g., along with a compatibilizer, to the reaction mixture such as for the production of a plastic. More particularly, most plastics contain organic base units that form repeating polymers. In most instances, these polymer units are based on chains of carbon (or silicon) atoms alone, but may include oxygen, sulfur, or nitrogen as well. The backbone is that part of the chain on the main “path” linking a large number of repeat units together. Additives can be added in a manner so as to be branched or otherwise hung off a portion of a monomer unit, such as prior to its forming of a polymer complex, so as to customize the properties of the plastic. For example, in various instances, an additive may be added to a monomer solution, prior to further processing of the solution, such as prior to the monomers being linked together to form the polymer backbone chain. The structure of these “side chains” may be designed so as to influence the properties of the polymer. Hence, in various instances, this fine tuning of the repeating unit's molecular structure influences the properties of the polymer and/or the plastics formed thereof.

Accordingly, various antimicrobial agents, such as fucoidan or its derivatives may be added during the fabrication process in such a manner that the resultant plastics contain these organic, and in some instances other inorganic, compounds that have been blended therein. The amount of the fucoidan and/or other additives may range such as from less than a percent, e.g., 0.5% or less, to five or ten percentage (for example in polymers used to wrap foods) to more than 50% for certain cosmetic and/or foam related applications. The average content of such additives may be about 2 to about 20% by weight of the polymer. Hence, a typical plastic may be formulated from a carbon or silicon containing base product, which may be reacted with other various chemical elements, such as hydrogen, oxygen, nitrogen, chlorine, and sulfur, as described herein. The carbon and/or silicon atoms may form up to four bonds with other constituents in such a manner that long strings or chains having a carbon backbone may be formed, and in some instances may form thermoplastics, such as where the plastic includes repeat units containing identical sub-units called monomers.

Such repeating monomers may be any suitable length so as to make a stable molecule, and in some instances may be only one carbon, e.g., with two hydrogens, in length, such as for polyethylene, to repeating monomers of 38 atoms in length, such as for nylon, and even more for other plastics. For instance, the formation of the repeat units, such as for thermoplastics may begin with the formation of small carbon-based molecules that can be combined to form monomers. The monomers, in turn, are joined together by chemical polymerization mechanisms to form polymers. These monomers are then chemically linked bonded into chains called polymers.

There are two basic mechanisms for polymerization: addition reactions and condensation reactions. For addition reactions, a special catalyst may be added, frequently a peroxide, so as to increase the speed and extent of the reaction, such as by causing or otherwise enhancing the ability of one monomer to bind or otherwise link to another and to the next and that to the next and so on, such as to form a string or chain of monomers. Such polymerization through addition may be used for the production of long chain polymers, including polyethylene, polystyrene, and polyvinyl chloride among others, without the creation of byproducts. In various instances, addition polymerization reactions are typically carried out in a gaseous phase, such as where the reactant monomers are dispersed in a liquid phase.

The second polymerization mechanism, condensation polymerization, also uses catalysts so as to have all the (single-unit cells) monomers present within a mixture of reactants to react with adjacent monomers so as to form dimers (e.g., two-unit cells). This reaction produces building blocks of two monomer cells that form dimers plus a byproduct. These dimers can then combine to form tetramers (four unit cells), which can then combine, and so on. For condensation polymerization the byproducts must be removed for the chemical reaction to produce useful products. Polyesters and nylons are typically made by condensation polymerization.

Further, when a connection or linkage between the carbon atoms is produced, a one dimensional chain of repeating building blocks (e.g., monomers, dimers, tetramers, etc.) is formed, so as to generate a chain of repeating unit cells. However, in various instances, two and/or three-dimensional networks may form, given the right fabrication conditions, instead of one-dimensional chains. In such an instance, the polymer that forms is a thermoset plastic, such as an epoxy or adhesive, which is characterized by not being meltable. In either instance, the polymer building blocks may be homopolymers or copolymers.

For instance, if the long chains show a continuous link of carbon-to-carbon atoms, e.g., of the same repeating monomer building blocks, each building block is called a homopolymer and forms a structure that is homogeneous. In such an instance, the long chain is called the backbone. Polypropylene, polybutylene, polystyrene and polymethylpentene are examples of polymers with a homogeneous carbon structure in the backbone. However, if the chains of carbon atoms are intermittently interrupted by other elements separating one carbon in the backbone from another, such as oxygen or nitrogen, the structure is called heterogeneous. Polyesters, nylons, and polycarbonates are examples of polymers with heterogeneous structure. Heterogeneous polymers as a class tend to be less chemically durable than homogeneous polymers.

As discussed herein, in various instances, different elements can be attached to the carbon-to-carbon backbone. For instance, one or more chemical elements may be added to the reaction mixture so as to enhance or otherwise modify the properties of the underlying plastic formed during the fabrication process. For instance, chlorine can be added during the reaction phase so as to produce polyvinyl chloride (PVC), which contains attached chlorine atoms. Additionally, fluorine can be added to the reaction mixture so as to form Teflon, which contains attached fluorine atoms. Likewise, in various instances, fucoidan, or one of its derivatives, e.g., sulfates, may be added to the reaction mixture, so as to make a plastic having or otherwise exhibiting antimicrobial properties.

In particular instances, how the links in the formation of the plastics, e.g., thermoplastic or otherwise, are arranged can be controlled so as to change the structure and properties of the underlying plastics, such as to modify the functionality of the products derived from the formulated plastics, such as to add antimicrobial properties thereto, such as by adding a fucoidan extract, e.g., sulfated polysaccharides, sulfated galactofucans, and/or sulfated galactans, during the reaction phase, or thereafter, to the forming or formed plastic, which can then function to depart antimicrobial properties to the plastic and/or a product produced from the resultant plastic. More particularly, some plastics are assembled from monomers such that there is intentional randomness in the occurrence of attached elements and chemical groups. In such an instance, fucoidan or a derivative thereof, may be added to the other various reactants during the fabrication process, so as to add one or more extracts, e.g., sulfonates, to the underlying carbon backbone or a branched chain thereof. So as the attached groups occur in a predictable order.

In other instances, the reaction may take place in such a manner so that the groups to be attached occur in a more randomized order. For example, once the fucoidan and/or one of its derivatives are isolated, dried, and formed into a powder, and/or the powder is processed into pre-selected size ranges, the powder may be added to the reaction mixture in such a manner that as the plastic monomers link to form the structural backbone the fucoidan element is incorporated into the underlying fabricated plastic. In alternative instances, the fucoidan extract may be formulated so as to be added to, e.g., coated upon, the finished plastic, such as after its fabrication.

In various instances, the reaction process and timing during which the various reactants are added to the reaction mixture, along with their concentration, may allow for the resultant plastic product to be specifically designed. For instance, by adjusting the spatial arrangement of atoms on the backbone chains, the plastics manufacturing process can change the performance properties of the plastic. Additionally, the chemical structure of the backbone, the use of copolymers, and the chemical binding of different elements and compounds to a backbone, and the use of crystallizability can change the processing, aesthetic, performance, and other properties of plastics. The plastics can also be altered by the inclusion of one or more additives, such as those herein described.

For example, when plastics emerge from the fabrication reactors in which they are produced, they may or may not have the properties desired for a commercial and/or industrial product. Particularly, dependent on the use to which these various products are to be used, it is often beneficial for them to have particular antimicrobial properties, such as properties that would allow the underlying product to exhibit anit-bacterail properties, such as properties that kill or otherwise prevent the growth and/or accumulation of harmful agents, such as deadly, injurious, and/or odor producing bacteria, and/or viruses. More particularly, as described herein, various additives, such as fucoidan and various of its extracts may be added to a reaction process, or to a finished product, so as to reduce about 80 to about 99% of the growth of gram positive and/or gram negative bacterial growth, such as Staphylococcus aureus and Escherichia coli. sulfated polysaccharides.

In such instances, the fucoidan may be part of the underlying composition or may be coated onto a surface of the product such as via a suitable treatment process. In particular instances, fucoidan and/or its extracts, such as sulfated polysaccharides, sulfated galactofucans, and/or sulfated galactans may be added to a composition, such as during a fabrication process, as described herein, or to a surface of a finished product, such as after fabrication so as to add anti-microbial and/or anti-viral properties thereto. Accordingly, in various instances, various additives may be included in the fabrication and/or manufacturing process so as to impart specific properties to plastics.

For instance, some of the monomers and/or polymers produced in accordance with the methods disclosed herein may incorporate additives during the manufacturing process. Other polymers include additives during processing into their finished parts. Hence, various additives, such as the fucoidan and/or its derivatives described herein, may be incorporated into the monomer and/or polymer production process so as to alter and improve the basic physical and/or chemical properties of the underlying products derived from the use of such plastics, such as in the manufacturing process. These additives may be used to protect the polymer and/or resultant plastics from the degrading effects of light, heat, or bacteria, and/or may be used to change such polymer processing properties such as melt flow, and/or to provide special characteristics such as improved surface appearance, reduced friction, and flame retardancy to the underlying product.

For example, fucoidan and/or other additives, may be added to the reaction mixture, such as to function as a filler, a plasticizer, a colorant, as an antimicrobial, and the like. Particularly, fucoidan may be added as a filler so as to improve performance and/or reduce production costs of the underlying plastic, e.g., to give the end product bulk and/or make the product cheaper by weight. It may also be added as a stabilizer and included as a fire retardant, such as to lower the flammability of materials. Many plastics contain fillers, relatively inert and inexpensive materials that. Other fillers may be mineral in origin, e.g., chalk, or may be more chemically active, e.g., zinc oxide, cellulose, starch, polysaccharides, so as to be a reinforcing agent. Plasticizers and/or plasticizer substitutes may also be added or otherwise blended with the reaction mixture so as to reduce the rigidity and/or increase the rheology of the underlying plastic to be produced. Additionally, various types of additives that may be incorporated may include antimicrobials, such as fucoidan and its derivatives, such as for plastic processing and outside application; antioxidants, e.g., where weathering resistance is desired; foaming agents, such as for expanded polystyrene cups and building board and for polyurethane carpet underlayment; plasticizers, such as used in wire insulation, flooring, gutters, and films; lubricants, such as used for making fibers, such as carpet fibers; anti-stats, so as to reduce dust collection by static electricity attraction; antimicrobials, e.g., sulfated polysaccharides, sulfated galactofucans, and/or sulfated galactans, such as used for shower curtains and wall coverings; and/or flame retardants, so as to improve the safety of wire and cable coverings and cultured marble.

As indicated, the underling plastic, e.g., to which the fucoidan additive may be added, may generally be a thermoplastic or a thermoset plastic. For instance, thermoplastics are plastics that do not undergo substantial chemical changes in its composition when heated and can be molded again and again, such as for the forming of a plastic substrate, which may be formed into a plastic containing end product. Examples of thermoplastics for use in accordance with the methods provided herein include polyethylene, polypropylene, polystyrene and polyvinyl chloride.

As described above, these plastics are formed from chains of organic molecules and are made up of many repeating molecular units, known as repeat units, derived from monomers which are linked to form polymers, which polymer chain may have up to several thousands of repeating units. In general, these thermoplastics may range from 20,000 to 500,000 amu. More particularly, a thermoplastic is a polymer in which the molecules are primarily held together by weak secondary bonding forces that soften when exposed to heat and return to its original condition when cooled back down to room temperature. For example, when a thermoplastic is softened by heat, it can be shaped and/or reshaped by many different methods, such as extrusion, molding, or pressing. Hence, thermoplastics offer versatility and a wide range of applications.

For instance, thermoplastics, such as those configured so as to include fucoidan, or an extract thereof, may be used for various products, such as in food packaging, health care products, cosmetics, and the like because they can be rapidly and economically formed into any shape needed to fulfill the designated functions. An example of thermoplastics include polyethylene, such as for packaging, e.g., for food packaging; films, e.g., such as plastic membranes; insulation; and other plastic substrates used in products, such as personal health care products, such as tooth brushes, hair brushes, combs, razors, dental floss, and the like. Another example includes polypropylene, which may be formed into various fibers, such as for the manufacture of various bristle or fibers, e.g., for use in bristle containing products, such as toothbrushes and hair brushes and carpets, as well as various containers, such as food containers, e.g., Tupperware®, electronic containers, such as mobile phone cases and housings, such as housings for various electronic devices. A further example includes polyvinyl chloride, such as for piping, sheathing, floor, wall, and counter covering, and the like.

Additionally, a thermoset plastic is a plastic that can melt and thereby be formed so as to take shape only once and then hardened into an immutable form. After they have solidified, they stay solid within the shape they were formed. Hence, in the thermosetting process, a chemical reaction occurs that is irreversible. Vulcanization is an exemplary thermosetting process. For instance, the vulcanization of rubber involves thermosetting such that before heating with sulfur, the polyisoprene is a tacky, slightly runny material, to which a fucoidan component may be added, but after vulcanization the product becomes rigid and non-tacky. Accordingly, a thermoset is a polymer that solidifies or “sets” irreversibly when heated or cured, and a thermoset polymer cannot be softened once “set”. Such thermosets are valued for their durability and strength and are used extensively in applications such as adhesives, inks, and coatings. Some examples of thermoset plastics to which an antimicrobial amount of fucoidan may be added and their product applications include: polyurethanes, such as for the production of mattresses, cushions, foams, and insulation; unsaturated polyesters, such as for clothing and fire-retardants; epoxies, for adhesives and glues, and phenol formaldehyde.

There are a variety of different fabrication and/or processing methods that may be employed so as to convert plastic monomers to polymers and/or then into finished products. These fabrication methods include extrusion, injection molding, rotational molding, compression molding, casting, thermoforming, and the like. For instance, extrusion is a continuous process that may be employed so as to produce films, sheets, profiles, tubes, pipes, and the like. In such a method, the plastic material and/or the additive, such as an antimicrobial fucoidan additive may be added together to form a mixture, such as in the form of granules, pellets, or powder, that may be loaded into a hopper and then fed into a long heated chamber through which the composition may be moved, such as by a conveyer, e.g., by the action of a continuously revolving screw.

The chamber is typically a cylinder referred to as an extruder. In various instances, extruders have one or two or more revolving screws. The plastic and additive composition may be melted by the mechanical work of the screw and the heat from the extruder chamber and/or walls. At the end of the heated chamber, the molten plastic is forced out through a small opening called a die to form the shape of the finished product. In certain instances, the additive, e.g., fucoidan and/or extracts thereof, may be added, such as in powder form, at this point. As the plastic is extruded from the die, it may be fed onto a conveyor apparatus, e.g., belt or rollers, and/or may be immersed in water for cooling. Examples of extruded products include various thermoplastics that are processed by continuous extrusion. Thermoset elastomer can also be extruded such as by adding catalysts to the rubber material as it is fed into the extruder.

Further, injection molding is a process that can produce intricate three-dimensional parts of high quality and great reproducibility. It is predominately used for thermoplastics but some thermosets and elastomers are also processed by injection molding. In injection molding, plastic and/or additive material may be fed into a hopper, which then feeds into an extruder. An extruder screw then pushes the plastic through a heating chamber in which the material is then melted so as to form a molten plastic. At the end of the extruder the molten plastic is forced under high pressure into a closed cold mold, for molding and cooling. In various instances, the additive, such as fucoidan and/or its extracts, may be added at this time. The high pressure helps ensure the mold is completely filled. Once the plastic cools, a solid substrate is produced. The mold may then be opened and the finished product is ejected. This process may be used to make such items as food containers, toys, e.g., infant toys, other containers, housings, e.g., housings for electronics, and the like.

In various instances, special catalysts can also be added to create the thermoset plastic products during the processing, such as cured carbon based and/or silicone rubber parts. Injection molding is a discontinuous process as the parts are formed in molds and must be cooled or cured before being removed. The economics are determined by how many parts can be made per cycle and how short the cycles can be.

In certain instances, rotational molding may be employed. For instance, rotational molding involves the use of a mold mounted on a machine that is configured for rotating on two or three axes simultaneously. Solid and/or liquid resin and or an additive, such as a fucoidan additive, may be placed within the mold and heat may then be applied. For example, rotational movement may be employed so as to distribute the plastic and/or the additive into a uniform coating on the inside of the mold. The mold may then be cooled until the plastic part cools and thereby hardens. This process may be used to make hollow configurations, such as shipping drums, storage tanks, as well as furniture and toys.

Compression molding may also be used such as for processing a prepared volume. For instance, the prepared volume of plastic and/or fucoidan and/or its extract additives may be placed into a mold cavity and then a second mold or plug may be applied so as to squeeze the plastic into the desired shape. The plastic can be a semi-cured thermoset, such as an automobile tire, or a thermoplastic, or a mat of thermoset resin and long glass fibers, such as for use as a plexi or fiberglass. In various instances, compression molding can be automated.

Transfer molding is a refinement of compression molding and may also be used for producing a plastic. Transfer molding is used to encapsulate parts, such as for semi-conductor manufacturing. Additionally, casting may also be employed for the fabrication of a plastic. Casting involves the use of low-pressure, e.g., pouring, of a resin and/or an additive into a mold. Catalyzed thermoset plastics can be formed into intricate shapes by casting. Furthermore, thermoforming may be used. For example, films of thermoplastic and/or an additive, such as fucoidan and/or an additive thereof, may be heated together so as to soften the film, and then the soft film may be pulled by vacuum or pushed by pressure to conform to a mold or pressed with a plug into a mold. In such instances, parts are thermoformed either from cut pieces for thick sheets, over 0.100 inches, or from rolls of thin sheet. Such finished parts may be cut from the sheet and the scrap sheet material recycled for manufacture of new sheet. The process can be automated for high volume production. These films, akin to saran wrap, may be used as wrappings, such as for the wrapping of food products.

Accordingly, in various instances, in accordance with the methods disclosed herein a plastic may be fabricated, such as where the plastic includes an additive, such as an antimicrobial additive, for instance, a fucoidan or component thereof. For instance, an amount of additive, e.g., fucoidan additive, may be added during the fabrication process, such as during and/or after fabrication, so as to impart antimicrobial properties to the plastic, and consequently to the end product that is made from the underlying plastic.

In particular instances, the antimicrobial to be added may be algae or an alga derived product. For instance, a salt water algae or fresh water algae may be added. Particularly, brown, red, and/or green algae, or an extract thereof may be added. More particularly, an algae extract, such as fucoidan may be added. In specific instances, the fucoidan is a fucoidan extract, such as sulfated polysaccharides, sulfated galactofucans, and/or sulfated galactans, which may be added to the reaction mixture, such as in the production of an antimicrobial plastic substrate and/or film or wrapper.

The amount of algae, e.g., brown algae, and/or an extract, and/or a component thereof, may be from 0.001% or less, or up to 50% or more, dependent upon the size, configuration, and use to which the end product is made, such as to provide antimicrobial properties thereto. For instance, for thin-film plastic and/or smaller container production, the amount of the additive to be added to the plastic precursors and/or to the fabricated plastic may be quite small, such as from 0.001% or less to about 5% or more, such as from about 0.01% to about 4%, for example, from about 0.1% to about 3%, including about 1% to about 2%. For other instances, such as in conjunction with the production of larger containers, and/or for the production of other plastic substrates for which increased antimicrobial properties may be desired, such as for medical and/or dental and/or other hygienic products, the amount of additive to added may be larger, such as from about 4% or about 5% to about 25% or about 30%, such as from about 7% to about 10% to about 20% or about 22%, for instance, from about 12% to about 17%, including about 15%. In some instances, the amount of algae and/or fucoidan and/or a component thereof to be added, such as in the production of a plastic may be even larger, such as from about 30% to about 50% per weight of plastic precursor to be added to the reaction mixture, such as for the production of an antimicrobial plastic. As indicated above, such plastics may be used such as for the production of food packaging products such as substrates and films, such as those used in the packaging of meat, meat products, fruits, vegetables, or other food product such as produce or other groceries, so as to give antimicrobial properties to the container or wrapping plastics that contain and/or wrap these products.

Accordingly, in one aspect, an apparatus may be provided, such as for containing or storing or otherwise contacting a perishable product, such as a meat, fruit, vegetable, or other consumable product, such as where the apparatus includes antimicrobial properties. In such an instance, a plastic or a plastic derived substrate may be provided, such as where the substrate has been fabricated or otherwise treated or coated in a manner so as to impart antimicrobial properties thereto. For instance, the plastic substrate may include an effective amount of an antimicrobial as recited herein, such as an effective amount of algae or an alga extract, such as fucoidan. Particularly, in various embodiments, an effective amount of one or more sulfated polysaccharides, sulfated galactofucans, and/or sulfated galactans on the plastic substrate so as to suppress bacterial growth, such as gram positive and/or gram negative bacteria, including Staphylococcus aureus and/or Escherichia coli, from growing on the plastic substrate.

As indicated, one or more of the antimicrobials detailed herein may be employed as an antimicrobial for use with a personal care product, such as a brush, a comb, a floss, and the like, such as for use with any personal health product made out of plastic. However, the use of such antimicrobial agents is not limited for use with plastics or plastic containing substrates. For instance, in some instances, the antimicrobial agents herein presented may be used with various non-plastic personal health items such as deodorants, lip-balms, facial creams, shaving creams, and the like.

Accordingly, in one embodiment, a deodorant composition may be provided. For example, a deodorant composition that includes an antimicrobial agent as herein described. Particularly, an emulsion may be provided, such as where the emulsion includes one or more of an aqueous phase, a fatty phase, and an effective amount of a microbial agent, such as a algae or algae extract, such as a fucoidan, or a fucoidan derivative, such as a sulfated polysaccharides, sulfated galactofucans, and/or sulfated galactans, so as to suppress a bacteria, such as an odor causing bacteria, for instance, a gram positive bacteria like Staphylococcus Aureus and/or gram negative bacteria like E. Coli, which may be present on the skin, such as in areas proximate the sweat glands of a person or animal.

Particularly, the antimicrobial agent may be a fucoidan, such as a fucoidan powder, which is extracted from brown algae, such as by using a water extraction process. In such an instance, the amount of the fucoidan extract, e.g., sulfated polysaccharide, in the emulsion may be between about 0.01 or about 0.05% to about 20 or about 30%, such as about 5% or 15%, including about 10%. In certain instances, the emulsion may be a water-in-oil emulsion or a water-in-silicone emulsion, and in particular instances, the deodorant composition may include a cosmetically acceptable fragrance.

Hence, in one aspect, a personal care product, such as a deodorant or antiperspirant may be provided, wherein the personal care product includes a fucoidan extract, so as to thereby include antimicrobial properties. Consequently, in various embodiments, a method of producing an antimicrobial deodorant and/or antiperspirant is provided. Particularly, an antiperspirant and/or deodorant (APD) stick is typically used to reduce underarm wetness and/or to control body odor. They are made by blending the active ingredients herein with waxes, oils, and silicones, and then molding the mixture into a roll-on, stick form.

For instance, body odor is generally generated in the area under the arms where there is a high concentration of sweat glands. While sweat from these glands may be odorless, it contains natural oils, e.g., lipids, which provide a growth medium for bacteria living on the skin. These bacteria interact with the lipids, converting them into compounds that have a characteristic foul odor. Isovaleric acid, amongst others, is a chemical compound produced by the interaction of the lipids discharged by the sweat glands with the bacteria inhabiting the body that gives sweat its smell. Primarily, there are two types of products used to control body odor.

The first, deodorants, reduce body odor by killing the odor-causing bacteria. These products do not affect the amount of perspiration the body produces. Antiperspirants, on the other hand, inhibit the activity of sweat glands so less moisture is produced. In addition to avoiding unpleasant wetness, these products also decrease odor because there is less sweat for the bacteria to act upon. Particularly, the antiperspirant salts from the APD stick form temporary plugs in some of the openings of the sweat glands so that the moisture is not secreted, thereby noticeably lessening underarm wetness. However, in both instances, the killing of the odor causing bacteria would be beneficial for reducing and/or preventing the under arm order caused by the secretions from the sweat glands.

With respect to antiperspirants, these typically include the active drug ingredients that control perspiration; gelling agents that form the stick matrix; and other ingredients, such as fragrance and/or colorants, which make the product aesthetically pleasing. Although these APD products have proven useful, they nevertheless may be benefitted from the addition of one or more of the antimicrobials herein presented, so as to better prevent and/or destroy the odor causing bacteria. Accordingly, in various instances, herein presented are novel antiperspirant and deodorant products that include an alga, such as a brown alga constituent, or an extract thereof. For instance, in particular embodiments, the various antimicrobial APD products provided herein may include a fucoidan or fucoidan component.

Particularly, in various embodiments, an effective amount of one or more sulfated polysaccharides, sulfated galactofucans, and/or sulfated galactans may be included in the ADP product. More particularly, the typical antiperspirant includes various active ingredients, such as aluminum chlorohydrate, aluminum chloride, aluminum sulfate, and aluminum zirconium (e.g., aluminum zirconium tetrachlorohydrex glycine) complexes such as for inhibiting or otherwise preventing the activity and/or growth of odor causing bacteria. However, these active agents contain metallic elements that are not particularly biodegradable and/or are not particularly environmentally friendly. The antimicrobial agents herein disclosed, conversely, are biodegradable and environmentally acceptable. And as such they may be used instead, e.g., in substitution, of the more harmful metal containing active agents, or in combination therewith.

Accordingly, in various embodiments, these algae derived materials may be supplied as powders, as described herein, for instance, at levels of about 1 or 2% to about 30 or about 40%, such as about 4 or about 5% to about 20 or about 25%, for instance, about 8 to about 15%, including about 10% based on the weight of the finished product. In such instances, the bulk of the formulation may include waxy or fatty materials that may be gelled to form a solid stick. Common examples include stearyl alcohol, cetyl alcohol, hydrogenated castor oil, and/or glyceryl stearate. These waxy materials may be blended with lubricating oils and emollients such as various silicone derived ingredients, such as cyclomethicone, which is a volatile silicone compound. These silicones are liquids at room temperature, but quickly evaporate thereby leaving the skin feeling smooth and dry. In addition, talc, starches, or other powders may be added to control stick consistency and to give the product a dry, smooth feel.

Other ingredients include fragrances and/or colorants that may be added to the formula, during the manufacturing process, so as to provide a pleasant odor or appearance. Accordingly, in various embodiments, a method of manufacturing a health care product, such as an antiperspirant and/or a deodorant having an algae derived antimicrobial is provided. In such instances, the method may include one or more steps such as batching, filling, and/or finishing. For instance, in the batching process, ingredients are combined in a suitable container, such as a jacketed stainless steel kettle. Heat, e.g., steam heat, may be applied to melt the ingredients while the batch is being mixed, so as to blend the mixture.

During this blending process, the temperature may be carefully controlled so as to avoid scorching the waxy ingredients. Once all the ingredients, including the algae derivatives have been added to the batch, it is blended until uniform. Once blended, the roll-on stick formation may be formed and filling may take place. Particularly, these APD stick packages are provided as hollow tubes with an elevator platform inside that is configured for moving the APD end product up and down so as to dispense the product. This platform can be configured in a variety of ways such that it is activated by being pushed up by hand, or it may be elevated such as by turning a screw that causes the platform onto which the end product is set to travel up along a central threaded post.

During production, these empty containers may be moved along a conveyor belt where the molten APD product is dispensed through a filling nozzle, e.g., from the top and/or bottom. Typically, the product is filled slightly above its congealing temperature so that it flows easily. If it is filled too hot, the dispersed solids may settle to the bottom; if it is filled too cold, air bubbles may be trapped in the stick. Once filled and cooled, various finishing operations may take place. The roll-on sticks may subjected to subsequent finishing operations to ensure the surface is smooth and that they are free from trapped pockets of air. These operations usually involve heating the sticks slightly by passing them under an infrared lamp. A probe may then be stuck into the center of the stick to allow air to escape and the surface is heated again to remelt the product, allowing it to flow into the void and thereby evacuate the air bubble. The APD sticks may then pass through a refrigeration unit, e.g., a tunnel, which rapidly lowers the temperature and causes them to solidify. Depending on the package design, a top or bottom piece is put into place to seal the container. These sticks may then pass through a cleaning stations prior to being placed in cartons for shipping.

As set forth herein, the present ADP preparations have been tested and it has been determined that the antiperspirants function reduces the amount of perspiration by at least 20% by a variety of test methods. This result may be confirmed such as by a visualization technique that shows the action of the sweat glands via a color change. Particularly, first, the skin to be tested is painted with a mixture of iodine castor oil and alcohol. After drying, the skin is then whitened with a layer of powdered starch. When sweat droplets are exuded, they appear as very dark spots against the white background. Applying an APD composition as herein described will amount to at least a 20% reduction in moisture production and/or exuding by the sweat glands

Another method that may be used to confirm this result may include painting a silicone polymer onto the skin to form a film. The temperature may be elevated thereby causing sweat to form. The film may then be peeled off and examined for tiny holes formed by the sweat drops. A relative measure of the amount of sweat produced by the body can be obtained by counting the number of holes in the film. Sweat production can also be measured using infrared gas sensors that detect moisture loss. In this process, a constant stream of gas is passed over the skin surrounding the sweat glands and is subsequently analyzed for moisture content. In either of these methods, it may be confirmed that the presence of sweat derived moisture is greatly reduced by the pre-application of an ADP product as provided herein to the skin proximate the sweat glands prior to sweating, such as caused by a rise in temperature or physical exertion. In various embodiments, the ADP stick herein may be configured in such a manner that the final antimicrobial ADP product forms a clear gel stick. In such an instance, the ADP product may be formulated as a gel, but may be formed as or otherwise dispensed from a stick-type form factor.

In addition to antimicrobial formulations for the production of plastics and personal health products, such as antiperspirants and deodorants, the algae derived antimicrobials herein presented may also be formulated as one or more cosmetics. Accordingly, in one aspect, an antimicrobial cosmetic composition is provided. For instance, in particular embodiments, the cosmetic composition may be a lipstick. Particularly, an antimicrobial lipstick and/or lip balms may be provided such as where the lipstick and/or lip balm composition includes various ingredients used to form the product to which the algae derivative herein disclosed is provided.

For instance, in one exemplary embodiment, a cosmetic composition for producing a lipstick and/or a lip balm is provided. The exemplary embodiment includes at least one liposoluble polymer, such as a liposoluble polymer having vinyl ester units. The composition may additionally include about 10% by weight of a propanol, such as 1-docosanoyloxy-3 (2-ethyl)-hexyloxy-2-propanol, or the like. Further, the composition may include at least one fatty body and/or a coloring agent, such as a non-toxic coloring material. Furthermore, so as to impart antimicrobial properties to the underlying lipstick formulation, an effective amount of an algae extract, such as a fucoidan or fucoidan extract, e.g., an effective amount of one or more sulfated polysaccharides, sulfated galactofucans, and/or sulfated galactans may be included so as to suppress the expression or activity of bacteria, such as a gram positive and/or gram negative bacteria, such as Staphylococcus aureus and/or E. Coli.

In particular instances, a sulfated polysaccharide may be added to the composition, such as where the sulfated polysaccharide is derived from fucoidan. For instance, the fucoidan and/or its extractant may be provided as a powder, e.g., a focoidan powder, which is extracted from brown algae, such as by using a water extraction process. Particularly, in various embodiments, these algae derived materials, e.g., including sulfated polysaccharides, may be supplied as powders, as described herein, for instance, at levels of about 0.001 or 0.01% to about 30 or about 40%, such as about 0.1% or about 0.5% to about 20 or about 25%, for instance, about 1 or 2 or about 5% to about 15%, including about 8 to about 10% based on the weight of the finished product.

In certain embodiments, the lipstick and/or lip balm composition may include a further cosmetic ingredient, such as selected from the group including a pearling agent, a perfume, an anti-solar agent, an anti-oxidant agent, a preservative and a solvent, such as for the insoluble coloring materials that may be present in the at least one fatty body. Accordingly, in various embodiments, a method of manufacturing a cosmetic care product, such as a lipstick or lip balm having an algae derived antimicrobial is provided. For instance, lipsticks and/or lip balms generally include dyes and pigments that have been combined with an oil-wax base, such as a fragranced oil-wax base. They are primarily encased within tubes that may be simple plastic screw type containers, similar to those described above with respect to APDs, such as for lip balms, or they may be more intricate and/or complex containers fabricated from more ornate metals, such as for lip sticks.

The size of the tube, and therefore the relative size of the lip stick and/or lip balm may vary, but is generally within the range of about 3 inches, e.g., about 10 cms, in length and about 0.5 inches, e.g., about 1 or 2 cms, in diameter. In particular instances, lip balms may be slightly less in these dimensions, particularly having a smaller diameter, such as 1 cm or less. The typical casing for lipsticks and lip balms includes two parts, a cover and a base. The base is made up of two components, a twisting or sliding portion that is configured for pushing or otherwise translocating the lipstick or lip balm up for application, and a container or tube like portion for containing the composition, to which the lid may be added so as to close the tube like container. The antimicrobial lipsticks and lip balms herein presented may be fabricated and encased within the above-described containers in any suitable fashion.

For example, the various raw lip stick and/or lip balm ingredients may first be melted separately, and then the oils and solvents along with an effective amount of the antimicrobial agents herein disclosed, e.g., a algae derived fucoidan extract, such as including one or more sulfated polysaccharides, sulfated galactofucans, and/or sulfated galactans, may be ground together with a desired color pigment(s). Particularly, after the pigment mass is prepared, it may be mixed with the hot wax. This mixture may then be agitated to free it of any air bubbles. Next, the antimicrobial lipstick and/or lip balm mixture may then be poured into tubing molds, cooled, and separated from the molds. It is to be noted that the antimicrobial composition may be applied, or reapplied at this step, such as by applying or otherwise coating the composition with an antimicrobial solution prior to its insertion into the tubular container.

Accordingly, as indicated the primary ingredients of lipsticks and lip balms are wax, oil, alcohol, and pigment, to which the antimicrobial agents herein disclosed are added. The wax component used may be any suitable, e.g., GRAS and/or FDA approved wax, such as beeswax, candelilla wax, or camauba wax, or a mixture thereof and/or with other waxes and/or thickening agents. This wax serves as a base to which the other ingredients are introduced, and thereby enables the mixture to be formed into the useful shapes for application to the lips. An oil component may also be included. Such oils may include mineral, caster, lanolin, or vegetable oils that are added to the wax.

Additionally, an antimicrobial component may be added such as in a bioeffective amount of algae derived materials may be included. Particularly, the composition of the end product may include sulfated polysaccharides, sulfated galactofucans, and/or sulfated galactans, which may be supplied as powders during the fabrication process, as described herein, for instance, at levels of about 0.001 or 0.01% to about 30 or about 40%, such as about 0.1% or about 0.5% to about 20 or about 25%, for instance, about 1 or 2 or about 5% to about 15%, including about 8 to about 10%. A pigment and/or fragrance may also be added, along with one or more preservatives and/or antioxidants, which prevent the lipstick and/or lip balm from becoming rancid. A wide variety of other ingredients can also be included to make the substance smoother or glossy or to moisten the lips.

It is to be noted that the proportions of these ingredients one to the other so as to form the final lipstick and/or lip balm composition may vary, but are generally include the following ranges. In general, the wax and oil components may make up about 60 percent of the lipstick (by weight), with alcohol and pigment and/or antimicrobial accounting for another 25 percent (by weight). In various instances, the antimicrobial portion may make up more that 25% such as more than 30 or 40%, however, in other instances, the antimicrobial may make up a mere fraction of a percent, and still be effective in imparting antimicrobial properties to the end product, as detailed herein. With respect to lipstick, a fragrance is typically added, but only accounts for one percent or less of the mixture. In addition to using lipstick to color the lips, there are also lip liners and pencils that may also include an antimicrobial as herein described.

Beyond these base ingredients (wax, oil, antimicrobials, and/or antioxidants) supplemental material amounts vary greatly. The ingredients themselves range from complex organic compounds to entirely natural ingredients, the proportions of which can determine various other characteristics of the lipstick or lip balm. More particularly, the manufacturing process, in detail, includes three basic steps: melting and mixing the lipstick, which may include the addition of an effective amount of antimicrobial agents at this stage; pouring the mixture into the tube, which also or alternatively include the addition of an effective amount of the antimicrobial agent at this stage; and packaging the product for sale. Since the lipstick and/or lip balm mass can be mixed and stored for later use, mixing does not have to happen at the same time as pouring. Once the lipstick and/or lip balm is in the tube, packaging for retail sale may be performed.

Accordingly, with respect to melting and mixing, first, the raw ingredients for the lipstick and/or the lip balm are melted and mixed, e.g., separately because of the different types of ingredients used. In such an instance, one mixture may contain the solvents, a second mixture may contain the oils, a third mixture may contain the fats and waxy materials, while a fourth mixture may contain the cosmetically and/or biologically effective amounts of the antimicrobial agents. However, it is noted that the antimicrobial materials may be added ton one or more of the previous mixtures, such as in powder form. These components are then heated in separate stainless steel or ceramic containers.

Likewise, the solvent solution and liquid oils may be mixed with the color pigments. If desired, a cosmetically and/or biologically effective amount of the antimicrobial agents may be added to this mixture as well as or in place of adding it to the above described components. This mixture is then passed through a roller mill, grinding the pigment to avoid a “grainy” feel to the lipstick. This process introduces air and/or the antimicrobial into the oil and pigment mixture, and as such mechanical working of the mixture is beneficial. The mixture may then be stirred for several hours, and/or a vacuum may be employed so as to withdraw air, such as air bubbles from the composition.

It is to be noted that where and when the antimicrobial is to be added to the composition may vary, but generally involve the addition of a fucoidan or fucoidan derived powder, such as a powder containing a cosmetically and/or biologically effective amount of sulfated polysaccharides, sulfated galactofucans, and/or sulfated galactans. For instance, the antimicrobial agents herein described may be obtained from algae, such as fresh and/or slat water algae, including brown, red, and/or green algae, from which fucos or a fucoidan extract may be obtained, which in turn may be further processed so as to derive an extract there form containing one or more of sulfated polysaccharides, sulfated galactofucans, and/or sulfated galactans. Such extractants may then be isolated and purified and formulated into a liquid, e.g., a liquid slurry, or a powder that may be dried and added to the various reaction mixtures herein described, such as for the production of various antimicrobial plastics, APDs, and cosmetics, such as for the inhibition of gram positive and/or gram negative bacterial growth, such as on a treated surface.

Particularly, as shown herein, the antimicrobial compositions herein disclosed are effective for reducing bacterial growth, e.g., S. aureus and/or E. coli, by about 80%, such as by about 85%, for instance, by about 90 or 95%, including about 99% or more, as compared to controls. After the pigment and/or antimicrobial mass is ground and mixed, it may be added to the hot wax mass until a uniform color and consistency is obtained. The fluid lipstick and/or lip balm may then be strained and molded, or it may be poured into pans and stored for future molding. For instance, the melt may be maintained at temperature, with agitation, so that trapped air escapes prior to being poured.

In various instances, these lipsticks and/or lip balms may be produced by hand or mass produced such as in a highly automated processes, at rates of up to 2,400 tubes an hour, or in essentially manual operations, at rates around 150 tubes per hour. The steps in the process basically differ only in the volume produced. Once the antimicrobial lipstick and/or lip balm mass is produced, it may then be molded into a desired, e.g., characteristically tubular, shape. For example, once the lipstick and/or lip balm mass is mixed and free of air, it is ready to be poured into the container, e.g., the tubular container.

In some instances, a high volume of batches may be run through a melter that agitates the lipstick and/or lip balm mass and maintains it as a liquid, such as at the desired mix temperature, with agitation. The melted mass is dispensed into a mold, which consists of the bottom portion of the metal or plastic tube and a shaping portion that fits snugly with the tube. The lipstick and/or lip balm is then poured “up-side down” so that the bottom of the tube is at the top of the mold. The composition is cooled (automated molds are kept cold; manually produced molds are transferred to a refrigeration unit) and separated from the mold, and the bottom of the tube is sealed. The antimicrobial lip protectant is then passed through a flaming cabinet (or is flamed by hand) to seal pinholes and improve the finish.

The finished product may then be packaged for sale. For instance, after the lipstick or lip balm is retracted and the tube is capped, the lipstick is ready for labeling and packaging. Packaging may or may not be highly automated, and the package used depends on the end use of the product rather than on the manufacturing process. It is to be noted that lipsticks and lip balms are generally the only cosmetics that may be ingested, and because of this strict controls on ingredients, as well as the manufacturing processes, are imposed. Lipstick and lip balm masses are mixed and processed in a controlled environment so they will be free of contamination, and incoming material is tested to ensure that it meets required specifications. Because of this, the organic algae derived antimicrobial materials here described are of particularly beneficial use for the production of antimicrobial cosmetics as herein described.

According to an embodiment of the present invention, there is provided a sulfated polysaccharide antimicrobial composition derived from renewable non-terrestrial plants that inhibits the growth of at least one of gram-negative bacteria Escherichia coli (E. Coli) and gram-positive bacteria Staphylococcus aureus (S. aureus) exceptionally well.

Experimental Section

Accordingly, as can be seen from the above, the antimicrobial compositions herein detailed are effective for reducing bacterial growth, e.g., S. aureus and/or E. coli, by about 80%, such as by about 85% growth, on a substrate or within a composition, for instance, by about 90 or 95%, including about 99% or more, as compared to controls. Particularly, various assays have been run, such as the Japanese Industrial Standard Test and the Kirby-Bauer assay, to determine the effectiveness of the herein presented antimicrobial formulations.

For instance, The Japanese Industrial Standard Committee (JIS) is an international organization that develops and standardizes test methods for a variety of products and materials. The JIS method Z 2801 is a quantitative test designed to assess the performance of antimicrobial finishes on hard, non-porous surfaces, such as plastics, such as the various plastics herein described above. The method can be conducted using contact times ranging from ten minutes up to 24 hours. For a JIS Z 2801 test, non-antimicrobial control surfaces are used as the baseline for calculations of microbial reduction. The method is versatile and can be used to determine the antimicrobial activity of a diverse array of surfaces including plastics, metals, and ceramics.

In this instance, test substances were first generated and contained: 0.5% antimicrobial (AM) and 85% Concentration of a plastic fabricated in accordance with the methods above. In this instance, the AM was coated onto the surface of the plastic. Other concentrations included: 1% AM and 15% Concentration; 1% AM 50% Concentration; 1.5% AM and 85% Concentration; 3% AM and 15% Concentration; 3% AM and 50% Concentration; 3% AM and 85% Concentration; and 6% AM and 15% Concentration; as well as 6% AM and 50% Concentration.

The first test microorganism selected was Staphylococcus aureus 6538. This bacterium is a gram positive, spherical-shaped, facultative anaerobe. Staphylococcus species are known to demonstrate resistance to antibiotics such as methicillin. S. aureus pathogenicity can range from commensal skin colonization to more severe diseases such as pneumonia and toxic shock syndrome (TSS), amongst other deadly diseases. S. aureus is commonly used in several test methods as a model for gram positive bacteria. It can be difficult to disinfect but does demonstrate susceptibility to low-level disinfectants.

Additionally, a second test microorganism selected was Escherichia coli 8739. This bacteria is a gram negative, rod shaped, facultative anaerobe commonly found in the gastrointestinal tract of mammals. Although most serotypes of this microorganism are harmless there are pathogenic groups of E. coli such as enterohemorrhagic (EHEC), verocytotoxin producing (VTEC) and Shiga-like toxin producing (STEC) that can cause a multitude of illnesses. E. coli is relatively susceptible to disinfection when dried on a surface, yet it can be a challenging microorganism to mitigate in solution.

In these test instances, the test microorganisms were grown in culture, the test and control surfaces were inoculated, the inoculated surfaces were incubated, and the test and control surfaces were evaluated after contact time and the percent log reductions, if any, were calculated. Particularly, the test microorganisms were independently prepared by growth in a liquid culture medium, such as Tryptic Soy Broth, for about 18 hours. The suspension of test microorganisms were then standardized by dilution, e.g., 1:500 Nutrient Broth, in a nutritive broth (to afford the microorganisms the opportunity to proliferate during the test). The control and test surfaces were inoculated, e.g., at a concentration of 4×105 CFU/Carrier, and a volume of 0.400 mL, with the microorganisms, and then the microbial inoculum is covered with a thin, sterile film. Covering the inoculum spreads it, prevents it from evaporating, and ensures close contact with the antimicrobial surface. The contact time was for about 24 hours at a temperature around 36 degrees C. The microbial concentrations were then determined at “time zero” by elution followed by dilution and plating to agar. A control was run to verify that the neutralization/elution method effectively neutralized the antimicrobial agent in the antimicrobial surface being tested. Inoculated, covered control and antimicrobial test surfaces were then allowed to incubate undisturbed in a humid environment for about 24 hours, usually at body temperature.

After incubation, the microbial concentrations were determined, and reduction of the microorganism relative to the control surface was calculated. The following calculations were performed: Percent reduction=(B-AB)×100, where: B=Number of viable test microorganisms on the control carriers after the contact time and A=Number of viable test microorganisms on the test carriers after the contact time; and Log₁₀ Reduction=Log (B/A), where B=Number of viable test microorganisms on the control carriers after the contact time, and A=Number of viable test microorganisms on the test carriers after the contact time. The results of these studies, tests 1 and 2, are set forth in Tables II and III:

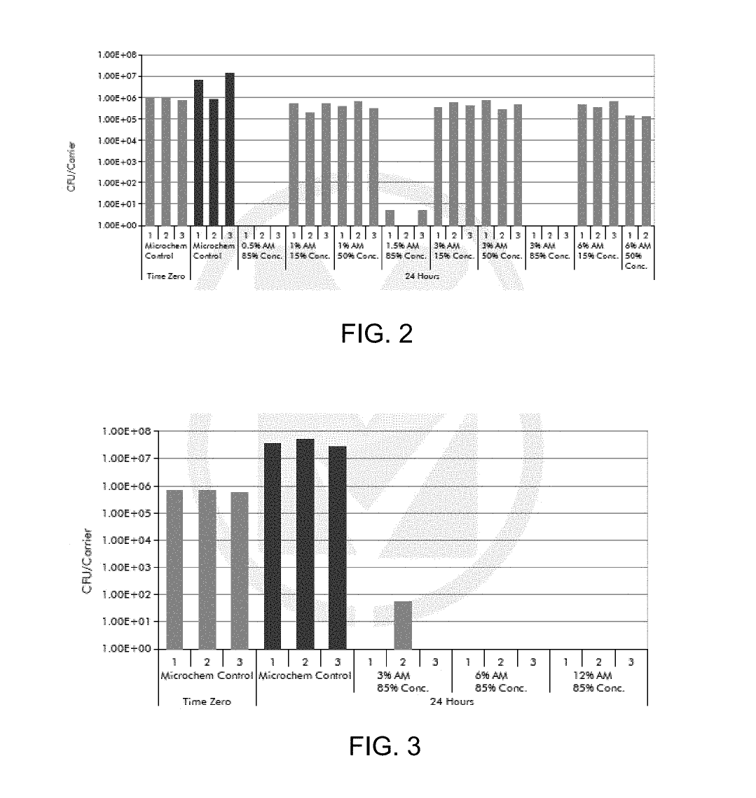

Additionally, these results may be seen with reference to FIGS. 2 and 3.

With respect to the various antiperspirants and/or deodratns disclosed hereon, the following tests were performed. For instance, a zone of inhibition test was performed. This test is a common microbiological method that is informative to the effectiveness of an antimicrobial product. The assay was qualitative, designed to visually demonstrate the inhibitory activity of water-diffusable antimicrobial agents, both in liquids and on treated objects. The Zone of Inhibition method was also used to assess antibiotic resistance of various microorganisms. Standards for antibiotic resistance testing are outlined by the Clinical and Laboratory Standards Institute (CSLI).

In this zone of inhibition assay, the treated materials were compared to known untreated and bacteriostatic controls as references for microbial growth inhibition. In particular, the test substance containing the antimicrobial fucoidan extract was formulated and tested. In a first instance, the test sample was an antimicrobial deodorant that was tested against a control sample deodorant. In this instance, the antimicrobial APD contained about 0.35% antimicrobial composition with an 85% concentration of deodorant base material, like that described herein above.

Like the first test microorganism above, S. aureus 6538 was selected. This bacterium is a Gram-positive, spherical-shaped, facultative anaerobe. Staphylococcus species are known to demonstrate resistance to antibiotics such as methicillin. S. aureus pathogenicity can range from commensal skin colonization to more severe diseases such as pneumonia and toxic shock syndrome (TSS), amongst other deadly diseases. S. aureus is commonly used in several test methods as a model for gram positive bacteria. It can be difficult to disinfect but does demonstrate susceptibility to low level disinfectants.

Particularly, the test antimicrobial APD composition was manufactured, the test organism, e.g., S. aureus at a concentration of 5.9×108 CFU/ml, was grown in culture and diluted, such as for about 18 to 24 hours. The culture was swabbed onto agar nutrient, e.g., Tryptic Soy Broth, plates and grown. The antimicrobial test substance, formulated in accordance with the methods herein described above, was applied to inoculated plates, the plates were then incubated, e.g., for about 14 hours at about 36 degrees C., and the zones of clear growth were measured. More particularly, the test microorganisms were prepared in liquid culture medium for bacteria or on agar for fungi. Suspensions of test microorganisms were standardized by dilution in a buffered saline solution. The test microorganisms were then swabbed onto prepared agar plates containing the appropriate growth agar, and care was taken to spread the bacterial and/or fungal cells over the entire agar plate. The prepared test substance was then placed in the center of the inoculated agar plate.

The agar plates were then incubated under appropriate conditions, and after incubation, large zones of clearance where microbial growth was inhibited appeared. These zones of inhibition were measured and reported. Specifically, a 1 mm zone of inhibition was observed around the test substance, and no observable zone of inhibition was seen around the control sample. Accordingly, in view of the above, the deodorant compositions as herein described are effective for inhibiting the growth and/or activity of bacteria, such as the odor producing bacteria present on the skin of the body around the sweat glands.

Although a few embodiments have been described in detail above, other modifications are possible. Other embodiments may be within the scope of the following claims.

1. A deodorant composition for a person, the deodorant composition comprising:
an emulsion comprising an aqueous phase, a fatty phase, and an effective amount of a sulfated polysaccharide to suppress Staphylococcus Aureus on skin areas proximate sweat glands of the person.

2. The deodorant composition in accordance with claim 1, wherein the sulfated polysaccharide is derived from fucoidan.

3. The deodorant composition in accordance with claim 2, wherein the focoidan is a focoidan powder extracted from brown algae using a water extraction process.

4. The deodorant composition in accordance with claim 1, wherein the amount of the sulfated polysaccharide in the emulsion is between 0.01 and 30 percent.

5. The deodorant composition in accordance with claim 4, wherein the amount of the sulfated polysaccharide in the emulsion is between 0.05 and 5 percent.

6. The deodorant composition in accordance with claim 1, further comprising a cosmetically acceptable fragrance.

7. The deodorant composition in accordance with claim 1, wherein the emulsion is a water-in-oil emulsion.

8. The deodorant composition in accordance with claim 1, wherein the emulsion is a water-in-silicone emulsion.

9. A cosmetic composition for lipsticks comprising:
at least one liposoluble polymer having vinyl ester units;
at least 10% by weight of 1-docosanoyloxy-3 (2-ethyl)-hexyloxy-2-propanol;
at least one fatty body;
at least one non-toxic coloring material; and
an effective amount of a sulfated polysaccharide to suppress Staphylococcus Aureus.

10. The cosmetic composition in accordance with claim 9, wherein the sulfated polysaccharide is derived from fucoidan.

11. The cosmetic composition in accordance with claim 10, wherein the focoidan is a focoidan powder extracted from brown algae using a water extraction process.

12. The cosmetic composition in accordance with claim 9, wherein the amount of the sulfated polysaccharide in the emulsion is between 0.01 and 30 percent.

13. The cosmetic composition in accordance with claim 12, wherein the amount of the sulfated polysaccharide in the emulsion is between 0.05 and 5 percent.

14. The cosmetic composition in accordance with claim 9, further comprising a cosmetic ingredient selected from the group consisting of a pearling agent, a perfume, an anti-solar agent, an anti-oxidant agent, a preservative and a solvent for the insoluble coloring materials in the at least one fatty body.

15. An apparatus comprising:
a substrate; and
an effective amount of a sulfated polysaccharide on the plastic substrate to suppress Staphylococcus Aureus and/or Escherichia coli proximate the substrate.

16. The apparatus in accordance with claim 15, wherein the substrate includes plastic.

17. The apparatus in accordance with claim 16, wherein the plastic includes 10 to 30 percent by weight of algae biomass.

18. The apparatus in accordance with claim 15, wherein the substrate includes expanded polystyrene.

19. The apparatus in accordance with claim 15, wherein the substrate is selected from the group of substrates that consist of: polyester (PES); polyethylene terephthalate (PET); polyethylene (PE); high-density polyethylene (HDPE); polyvinyl chloride (PVC); polyvinylidene chloride (PVDC); low-density polyethylene (LDPE); polypropylene (PP); polystyrene (PS); high impact polystyrene (HIPS); polyamides (PA); acrylonitrile butadiene styrene (ABS); polyethylene/acrylonitrile butadiene styrene (PE/ABS); polycarbonate (PC); polycarbonate/acrylonitrile butadiene styrene (PC/ABS); and polyurethanes (PU).

20. The apparatus in accordance with claim 15, wherein the substrate includes sugar-cane derived plastic.
